(19) United States
(12) Patent Application Publication
(10) Pub. No.: US 2021/0130411 A1
(43) Pub. Date: May 6, 2021

(54) CYCLIC HEXAPEPTIDES COMPOUNDS WITH ANTI-MALARIAL ACTIVITY

(71) Applicants: Stella PEÑA BARÓN, Montevideo (UY); UNIVERSIDAD DE LA REPÚBLICA (UDELAR), Montevideo (UY)

(72) Inventors: Gloria SERRA LEMES, Montevideo (UY); Stella PEÑA BARÓN, Montevideo (UY); Catherine FAGUNDEZ OLIVENCIA, Montevideo (UY); Laura SCARONE ZAPATA, Montevideo (UY); Diver Sellanes Fernández, Montevideo (UY)

(21) Appl. No.: 17/050,405

(22) PCT Filed: Apr. 16, 2019
(86) PCT No.: PCT/IB2019/053125

§ 371 Date: Oct. 23, 2020

Related U.S. Application Data
(60) Provisional application No. 62/662,275, filed on Apr. 25, 2018.

(51) Int. Cl. C07K 7/64; A61P 33/06

(52) U.S. Cl. CPC C07K 7/64; A61K 38/00; A61P 33/06

(57) ABSTRACT

The present invention relates generally to compositions for medical treatment of malaria. In particular, the present invention relates to novel cyclic hexapeptides compounds of formula (I) with anti-malarial activity, a method for the synthesis of said compounds and pharmaceutical compositions containing said cyclic hexapeptides compounds which are useful for treating malaria.

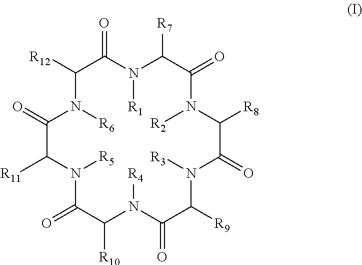

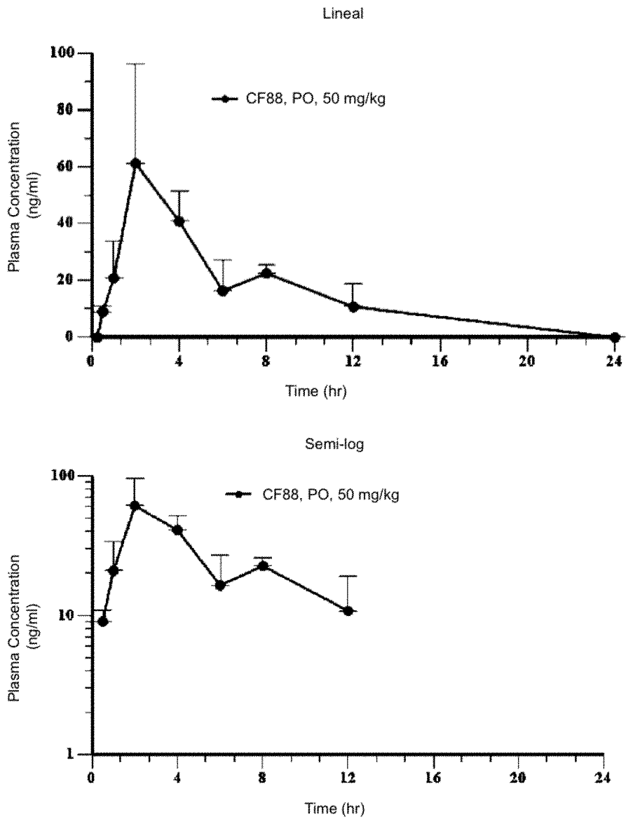

### TECHNICAL FIELD

The present invention relates generally to compositions for medical treatment of malaria. In particular, the present invention relates to novel cyclic hexapeptides compounds with anti-malarial activity, a method for the synthesis of said compounds and pharmaceutical compositions with said cyclic hexapeptides compounds useful for treating malaria.

### BACKGROUND OF THE INVENTION

Parasitic tropical diseases such as Human African Trypanosomiasis (HAT) and malaria affect almost 300 million people every year. Malaria is a life-threatening illness caused by Plasmodium parasites that is transmitted to human through the bite of an infected female of the Anopheles mosquito. In particular, P. falciparum is the deadliest of the four species that cause malaria. The symptoms of malaria comprise headache, lassitude, fatigue, abdominal discomfort, muscle and joint aches, usually followed by fever, chills, perspiration, anorexia, vomiting and worsening malaise, and the patient may develop potentially lethal severe malaria (WHO. Guidelines for the treatment of malaria. Third edition. April 2015).

The standard treatment for malaria includes an artemisinin-based combination therapy. Nevertheless, it is a major problem the emergence of drug resistance malaria parasites and it has been documented the resistance to all classes of antimalarial medicines, including the artemisinin derivatives. Therefore, over the last decade, several research groups have been involved in the isolation, design and synthesis of new anti-parasitic compounds.

Several cyclic peptides displaying anti-parasitic activity have been identified over the last few years. For example, Linington and co-workers have isolated antimalarial venturamides from marine cyanobacterium, which show an effect against the P. falciparum W2 chloroquine-resistant strain, and a mild cytotoxicity to mammalian Vero cells (Linington R G, Gonzalez J, Ureña LD, Romero L I, Ortega-Barria E, Gerwick W H. Venturamides A and B: Antimalarial Constituents of the Panamanian Marine Cyanobacterium Oscillatoria sp. J. Nat. Prod., 2007, 70 (3), pp 397-401). Portmann and co-workers have isolated aerucyclamides from the cyanobacterium Microcystis aeruginosa PCC 7806, which show promising antiplasmodial activity towards the P. falciparum K1 chloroquine-resistant strain (Portmann C, Blom J F, Gademann K, Jüttner F. Isolation of Aerucyclamides C and D and Structure Revision of Microcyclamide 7806A: Heterocyclic Ribosomal Peptides from Microcystis aeruginosa PCC 7806 and Their Antiparasite Evaluation. J. Nat. Prod., 2008, 71 (11), pp 1891-1896). Baraguey and co-workers have isolated cyclic peptides Mahafacyclin and Chevalierin from the latex of Jatropha species that showed an interesting effect against P. falciparum (Baraguey C, Blond A, Cavelier F, Pousset J L, Bodo B, Auvin-Guette C. Isolation, structure and synthesis of mahafacyclin B, a cyclic heptapeptide from the latex of Jatropha mahafalensis. J. Chem. Soc., Perkin Trans. 1, 2001, 17, pp 2098-2103; Baraguey C, Auvin-Guette C, Blond A, Cavelier F, Lezenven F, Pousset J L, Bodo L. Isolation, structure and synthesis of chevalierins A, B and C, cyclic peptides from the latex of Jatropha chevalieri. J. Chem. Soc., Perkin Trans. 1, 1998, 18, pp 3033-3040).

Additionally, several patent documents also disclose cyclic peptides with anti-fungal, anti-protozoal and anti-microbial activity, such as the patents No. U.S. Pat. Nos. 5,229,363, 6,232,290, 9,133,237 and the patent applications No. WO 2000/020441, EP 0943623, EP 0494515, US 2017/0232110.

Although, the compounds disclosed in the cited publications exhibit activity against Plasmodium sp., their EC50 (effective concentration to kill 50% of the parasites) are in the micro molar or 100 nano molar order. Said compounds are much less active than artemisinin (EC50=20 nM) and artesunate (EC50=5 nM), therefore efforts have being made to make structural variations in cyclohexapeptides in order to improve their activity. In this direction, cyclohexapeptides containing N-methylated amino acids have shown to possess interesting structural characteristics, such that they allow the structures to adopt suitable conformations to permeate membranes. This could evidently increase the biological activity; therefore the N-methylated cyclohexapeptides were synthesized.

Thus, since the current compounds available for treating malaria are not effective due to poor efficacy, undesirable route of administration, side effects or unacceptable toxicity, given the number of people infected and the increase in resistance of malarial parasites, there is a need for new safe and effective anti-malarial drugs.

### SUMMARY OF THE INVENTION

In one aspect of the present invention, the cyclic hexapeptide compounds provided comprise the following general formula (I):

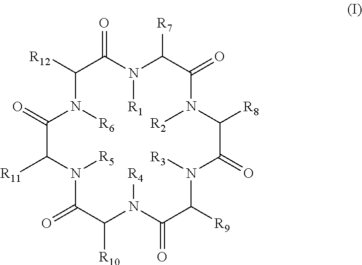

or any salt, solvate, prodrug, stereoisomer, tautomer thereof, wherein
R₁, R₂, R₃, R₄, R₅ and R₆ are independently selected from methyl (—CH₃) and hydrogen (—H);
R₇ is hydrogen (—H), CH₃ or isopropyl.
R₈ and R₁₀ are independently selected from CH₂SC(C₆H₅)₃, CH₂SCH₃, CH₂SH, CH₂S—SR₁₃ (R₁₃═CH₃ or cysteine derived), represented by the formulas:

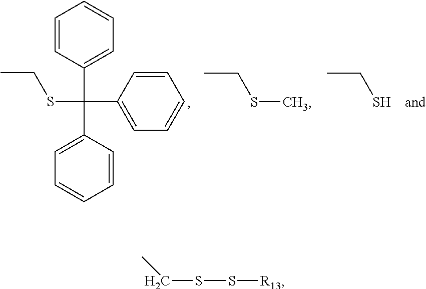

respectively
R₉ and R₁₁ are independently selected from CH(CH₃)CH₂CH₃, CH₂C₆H₅, CH₃, CH₂CH₂SCH₃ and H, represented by the formulas:

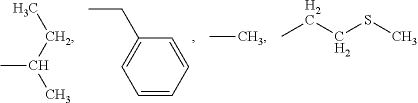

and H′ respectively
R₁₂ is selected from CH₂OH, CH(CH₃)OH, CH₂OC(CH₃)₃, (CH₂)₂COOH, CH(CH₃)OC(CH₃)₃, (CH₂)₂COOR₁₄ represented by the formulas:

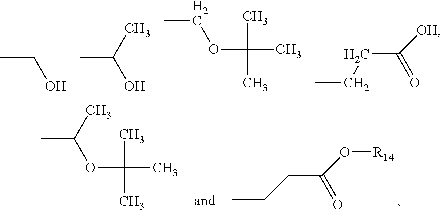

respectively
wherein R₁₄ is an alkyl group (example: CH₃, —CH₂CH₃, n-butyl, C(CH₃)₃, n-propyl, CH(CH₃)₂, etc.).

In one embodiment, the present invention provides cyclic hexapeptides according to the formula (I), wherein at least one of R₁, R₂, R₃, R₄, R₅, R₆ is a methyl group (—CH₃).

In a preferred embodiment, the present invention provides cyclic hexapeptides according to the formula (I), which is selected from the following compounds:

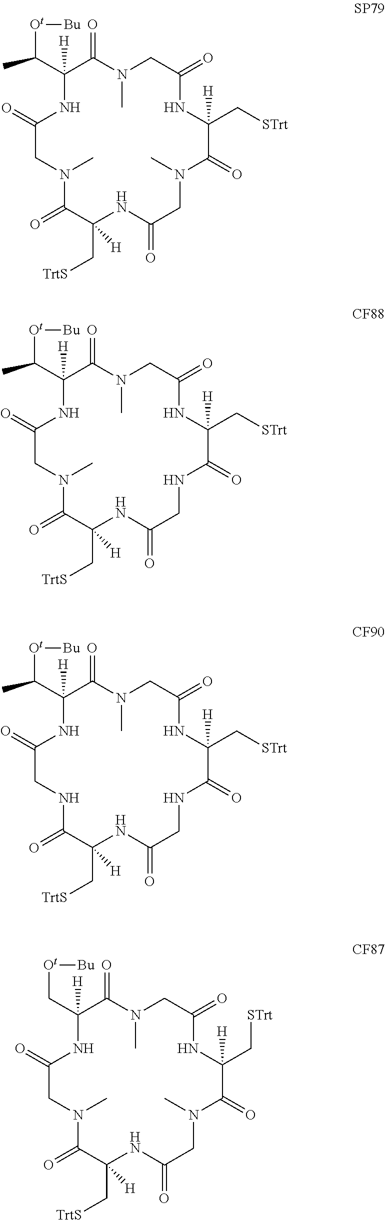

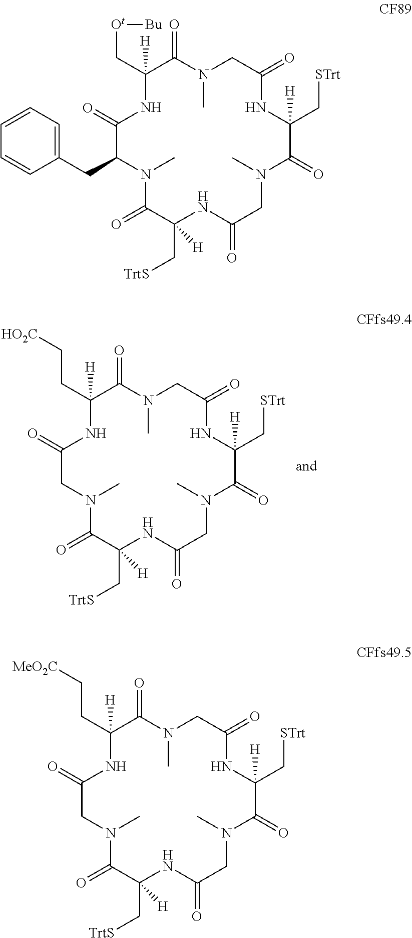

or
any salt, solvate, prodrug, stereoisomer, tautomer thereof, wherein

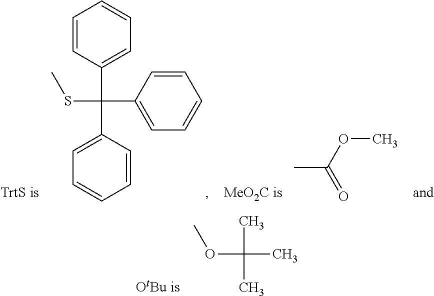

In a further preferred embodiment, the present invention provides cyclic peptides according to the formula (I), which is CF88 represented by the formula:

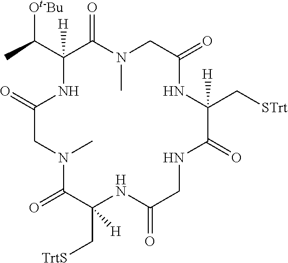

In a further preferred embodiment, the present invention provides cyclic peptides according to the formula (I), which is CFfs49.4 represented by the formula:

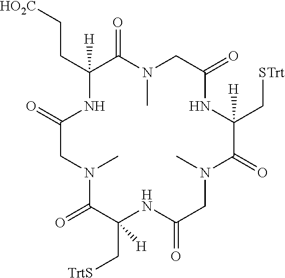

In a second aspect, the present invention provides a pharmaceutical composition for the treatment of malaria, containing a cyclic hexapeptide compound according to the general formula (I) mentioned above or any salt, solvate, prodrug, stereoisomer, tautomer thereof, and a pharmaceutically acceptable excipient. In a preferred embodiment, said pharmaceutical composition contains the cyclic hexapeptide with the general formula (I), or any salt, solvate, prodrug, stereoisomer or tautomer thereof, wherein:
R₁, R₂, R₃, R₄, R₅ and R₆ are independently selected from methyl (—CH₃) and hydrogen (—H);
R₇ is hydrogen (—H), CH₃ or isopropyl.
R₈ and R₁₀ are independently selected from CH₂SC(C₆H₅)₃, CH₂SCH₃, CH₂SH, CH₂S—SR₁₃ (R₁₃═CH₃ or cysteine derived), represented by the formulas:

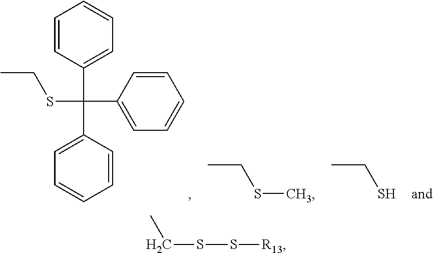

respectively
R₉ and R₁₁ are independently selected from CH(CH₃)CH₂CH₃, CH₂C₆H₅, CH₃, CH₂CH₂SCH₃ and H, represented by the formulas:

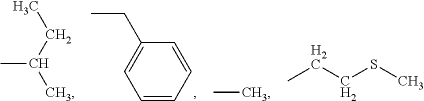

and H, respectively
R₁₂ is selected from CH₂OH, CH(CH₃)OH, CH₂OC(CH₃)₃, (CH₂)₂COOH, CH(CH₃)OC(CH₃)₃, (CH₂)₂COOR₁₄ represented by the formulas:

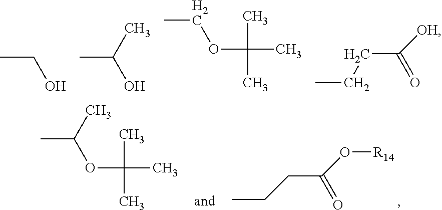

respectively
wherein R₁₄ is an alkyl group (example: CH₃, —CH₂CH₃, n-butyl, C(CH₃)₃, n-propyl, CH(CH₃)₂, etc.), and a pharmaceutically acceptable excipient.

In a further preferred embodiment, said pharmaceutical composition contains the cyclic hexapeptide with the general formula (I), wherein at least one of R₁, R₂, R₃, R₄, R₅ or R₆ is a methyl group (—CH₃).

In a further preferred embodiment, said pharmaceutical composition contains the cyclic hexapeptide with the general formula (I), which is selected from the following compounds:

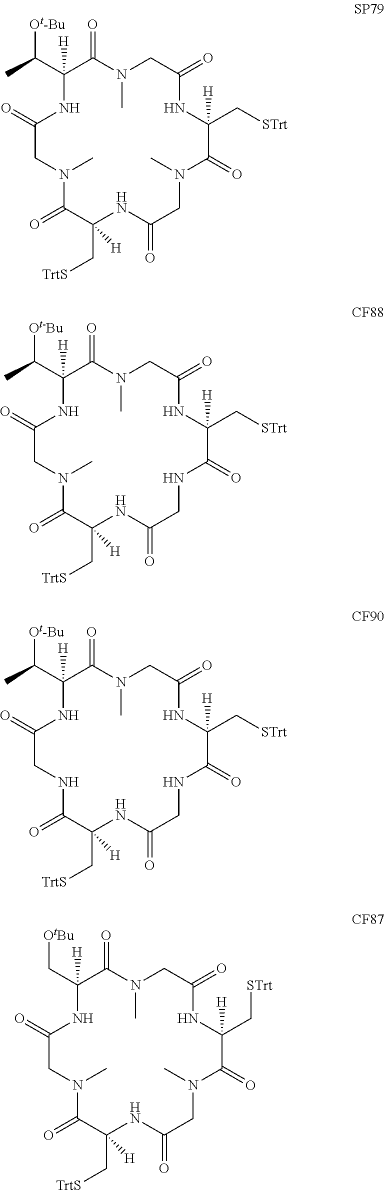

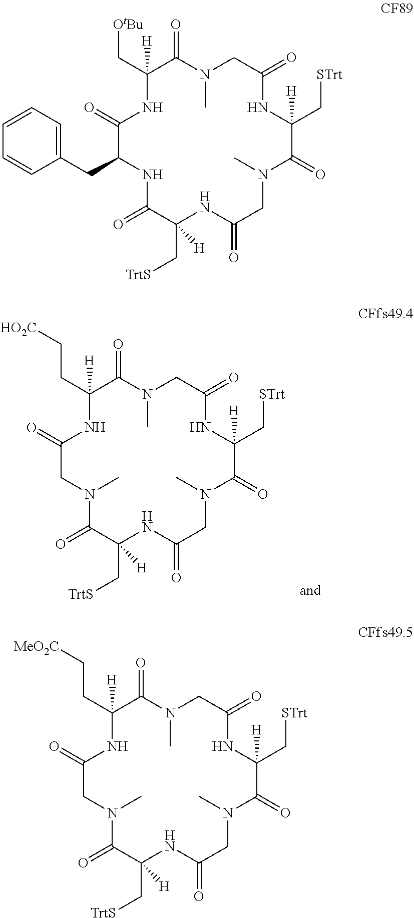

or any salt, solvate, prodrug, stereoisomer, tautomer thereof.

In a preferred embodiment, the pharmaceutical composition for the treatment of malaria contains the compound CF88, represented by the formula:

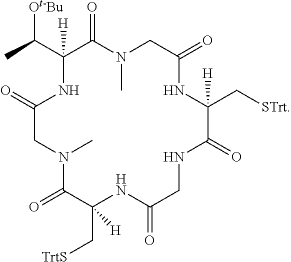

In a further preferred embodiment, the pharmaceutical composition for the treatment of malaria contains the compound CFfs49.4, represented by the formula:

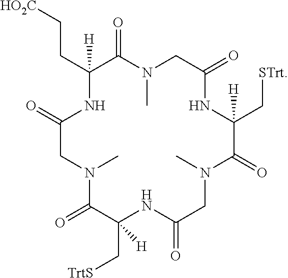

In a third aspect, the present invention provides a method for the treatment of malaria, comprising administering to a patient a pharmaceutical composition containing a cyclic hexapeptide according to the general formula (I) mentioned above, or any salt, solvate, prodrug, stereoisomer or tautomer thereof, wherein:

R₁, R₂, R₃, R₄, R₅ and R₆ are independently selected from methyl (—CH₃) and hydrogen (—H);
R₇ is hydrogen (—H), CH₃ or isopropyl.
R₈ and R₁₀ are independently selected from CH₂SC(C₆H₅)₃, CH₂SCH₃, CH₂SH, CH₂S—SR₁₃ (R₁₃═CH₃ or cysteine derived), represented by the formulas:

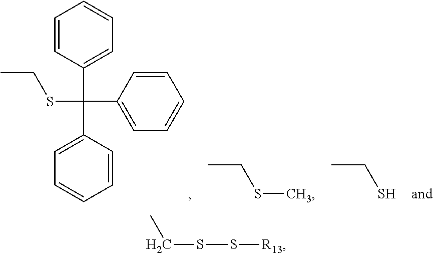

respectively
R₉ and R₁₁ are independently selected from CH(CH₃)CH₂CH₃, CH₂C₆H₅, CH₃, CH₂CH₂SCH₃ and H, represented by the formulas:

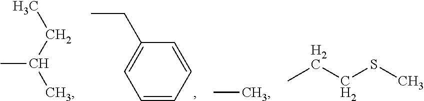

and H′ respectively
R₁₂ is selected from CH₂OH, CH(CH₃)OH, CH₂OC(CH₃)₃, (CH₂)₂COOH, CH(CH₃)OC(CH₃)₃, (CH₂)₂COOR₁₄ represented by the formulas:

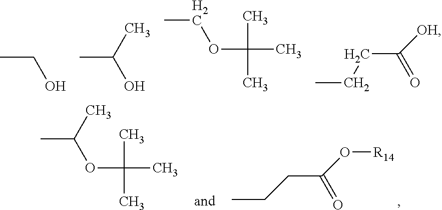

respectively
wherein R₁₄ is an alkyl group (example: CH₃, —CH₂CH₃, n-butyl, C(CH₃)₃, n-propyl, CH(CH₃)₂, etc.).

In a further preferred embodiment, said pharmaceutical composition administered to a patient contains the cyclic hexapeptide with the general formula (I), wherein at least one R₁, R₂, R₃, R₄, R₅, R₆ is a methyl group (—CH₃).

In a further preferred embodiment, said pharmaceutical composition administered to a patient contains the cyclic hexapeptide with the general formula (I), which is selected from the following compounds:

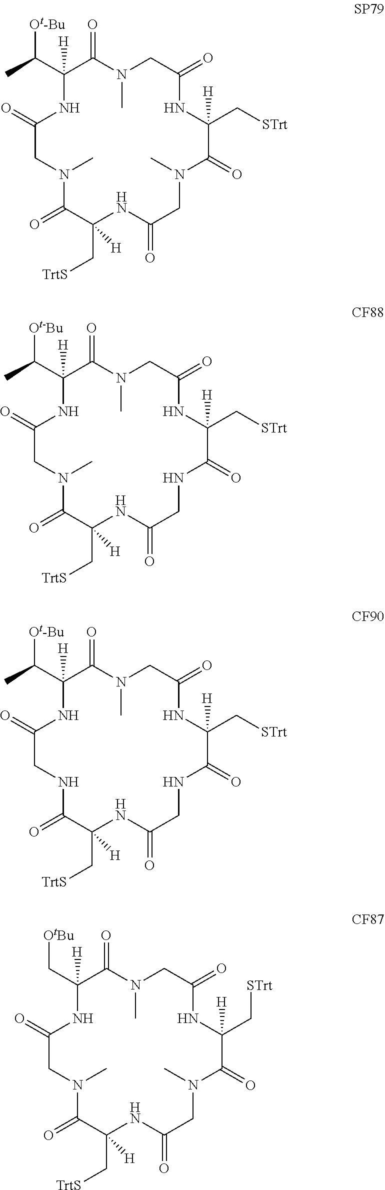

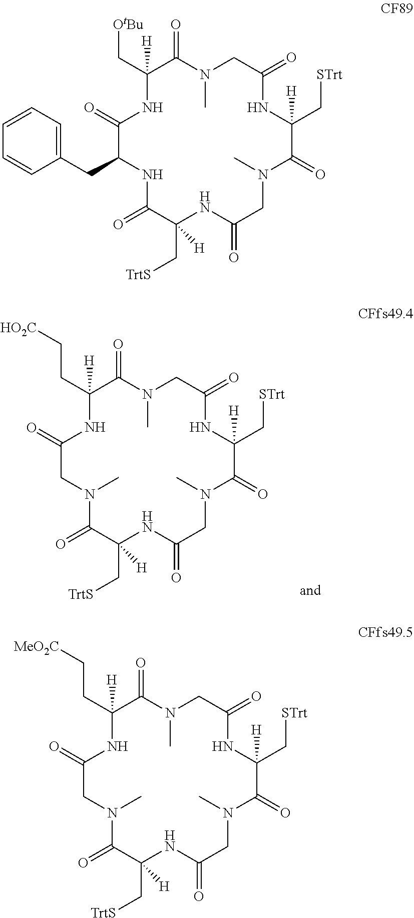

or any salt, solvate, prodrug, stereoisomer, tautomer thereof.

In a preferred embodiment, the pharmaceutical composition administered to a patient for the treatment of malaria contains the compound CF88, represented by the formula:

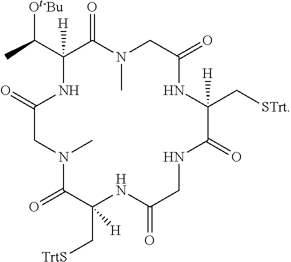

In a further preferred embodiment, the pharmaceutical composition administered to a patient for the treatment of malaria contains the compound CFfs49.4, represented by the formula:

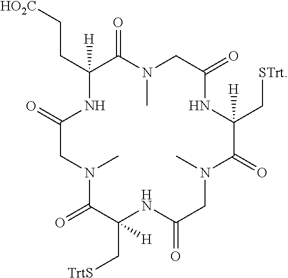

In a further preferred embodiment, the method for the treatment of malaria, comprising administering to a patient the pharmaceutical composition previously mentioned for at least 3 days, preferably administered orally.

### BRIEF DESCRIPTION OF THE DRAWINGS

The sole FIGURE shows the profile of mean plasma concentration vs time of compound CF88 with one oral administration in male Swiss Albino mice (dose: 50 mg/Kg).

### DETAILED DESCRIPTION OF THE INVENTION

The present invention provides novel cyclic hexapeptides compounds for treating malaria, a pharmaceutical composition containing said compounds and a method of treatment of malaria that comprises administer said pharmaceutical composition to a patient.

These novel cyclic hexapeptides have high enhanced anti-malarial activity and an outstanding selectivity against malarial parasites, in particular, P. falciparum, compared with the current available drug treatments. In addition, these compounds have an excellent activity against the two forms of the parasite: hepatic and intraerythrocytic and have shown to be nontoxic for hepatic cells and macrophages. In consequence, the cyclic hexapeptides disclosed herein provides a safe and effective alternative for treating malaria.

All technical and scientific terms used to describe the present invention have the same meaning understood by a person having a basic knowledge in this technical field. Notwithstanding, to define the scope of the invention more clearly, a list of terminology used in this description is included below.

It should be understood that as used herein, the term “anti-parasitic” or “anti-malarial activity” includes preventing, stopping, retarding, alleviating, ameliorating, halting, restraining, slowing or reversing progression, or reducing the severity of the growth or any attending characteristics, symptoms, and results from the existence of the malaria parasite. As such, the method of treatment of malaria include both medical therapeutic (acute) and/or prophylactic (prevention) administration as appropriate.

It should be understood that as used herein, the term “active ingredient” is referred to a cyclic hexapeptide compound with the general formula (I), any of the variants describe herein or pharmaceutical or any salt, solvate, prodrug, stereoisomer, tautomer thereof.

It should be understood that as used herein, the term “pharmaceutical acceptable salts” concerns to salts of the cyclic hexapeptide compounds of the general formula (I) which, at the doses administered, are non-toxic to living organisms. Typical pharmaceutical salts include those prepared by reaction of the compounds of the present invention with organic or inorganic bases. The same applies to any prodrug, stereoisomer or tautomer of said active ingredient.

It should be understood that as used herein, the term “solvate” represents an aggregate that comprises one or more molecules of the solute, such as the cyclic hexapeptide compounds of the general formula (I), with one or more molecules of solvent or solvant. Said solvents may be any solvent, or mixture of solvents, inert to the ongoing reaction that sufficiently solubilizes the reactants. Said solvant may be any compound that enhances the solubility of the cyclic hexapeptides.

It should be understood that as used herein, the term “effective amount” means an amount of a cyclic hexapeptide compound of the general formula (I), which is capable of having anti-parasitic or anti-malarial activity.

In one aspect, the cyclic hexapeptide compounds provided by this invention comprise the following general formula (I):

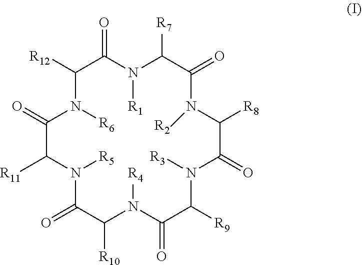

or any salt, solvate, prodrug, stereoisomer, tautomer thereof, wherein
R₁, R₂, R₃, R₄, R₅ and R₆ are independently selected from methyl (—CH₃) and hydrogen (—H);
R₇ is hydrogen (—H), CH₃ or isopropyl.
R₈ and R₁₀ are independently selected from CH₂SC(C₆H₅)₃, CH₂SCH₃, CH₂SH, CH₂S—SR₁₃ (R₁₃═CH₃ or cysteine derived), represented by the formulas:

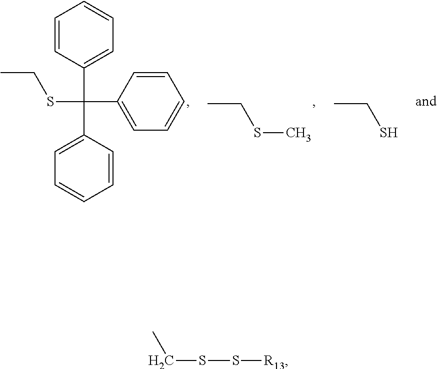

respectively
R₉ and R₁₁ are independently selected from CH(CH₃)CH₂CH₃, CH₂C₆H₅, CH₃, CH₂CH₂SCH₃ and H, represented by the formulas:

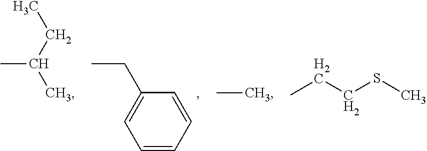

and H′ respectively
R₁₂ is selected from CH₂OH, CH(CH₃)OH, CH₂OC(CH₃)₃, (CH₂)₂COOH, CH(CH₃)OC(CH₃)₃, (CH₂)₂COOR₁₄ represented by the formulas:

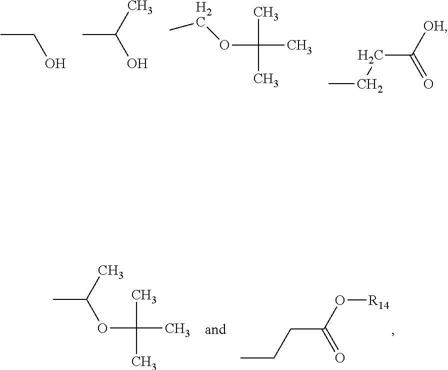

respectively
wherein R₁₄ is an alkyl group (example: CH₃, —CH₂CH₃, n-butyl, C(CH₃)₃, n-propyl, CH(CH₃)₂, etc.).

In one embodiment, the present invention provides cyclic hexapeptides according to the formula (I), wherein at least one of R₁, R₂, R₃, R₄, R₅ or R₆ is a methyl group (—CH₃).

Exemplary cyclic hexapeptide compounds which shown acceptable anti-malarial activity are the following:

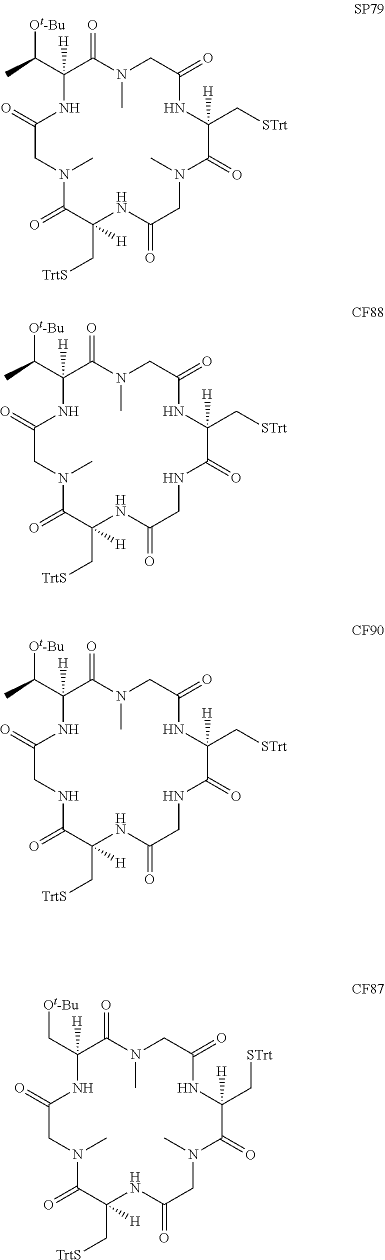

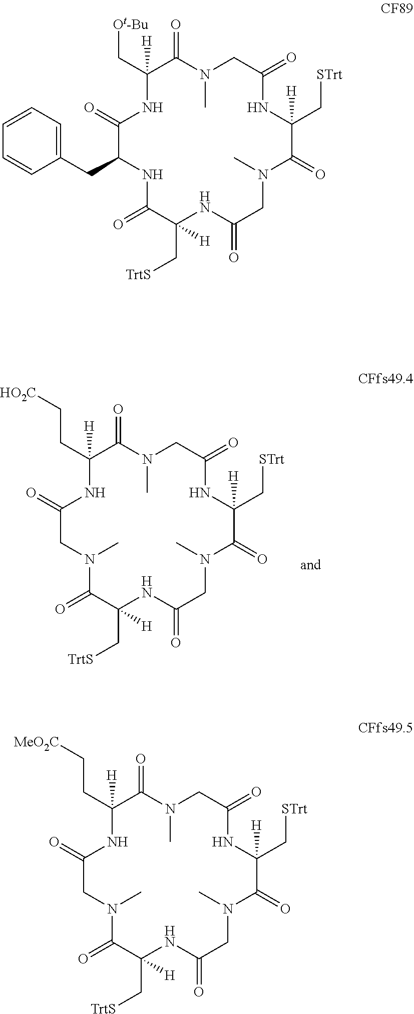

or
any salt, solvate, prodrug, stereoisomer, tautomer thereof.

In a preferred embodiment, the present invention provides cyclic hexapeptides according to the formula (I), which is CF88 represented by the formula:

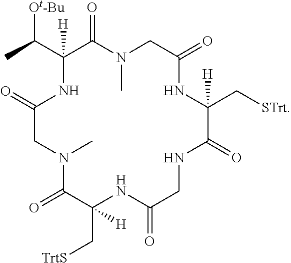

In a further preferred embodiment, the present invention provides cyclic peptides according to the formula (I), which is CFfs49.4 represented by the formula:

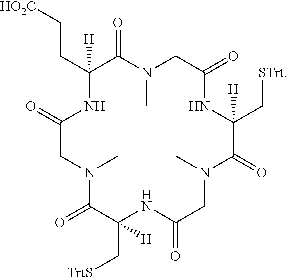

All mentioned cyclic hexapeptides are useful as anti-parasitic agents, especially useful as anti-malarial agents, or as intermediates to such agents.

The synthesis of the cyclic hexapeptide compounds of the general formula (I), may be prepared by Fmoc-based solid phase peptide synthesis (SPPS), followed by macrocyclation either on-resin or in solution, as illustrated in Scheme 1 below, wherein Method A refers to the synthesis of the peptide sequence on resin, followed by the cleavage and macrocyclization in solution, whereas Method B refers to synthesis and macrocyclation both performed on-resin. TFA is trifluoroacetic acid, DMF is dimethylformamide, DCM is dichloromethane, HBTU is 3-[bis(dimethylamino)methyliumyl]-3H-benzotriazol-1-oxide hexafluorophosphate, HATU is 1-[bis(dimethylamino)methylene]-1H-1,2,3-triazolo[4,5-b]pyridinium 3-oxide hexafluorophosphate, DMAP is 4-dimethylaminopyridine, DIPEA is N,N-diisopropylethylamine, Pd(PPh₃)₄ is tetrakis(triphenylphosphine)palladium(0).

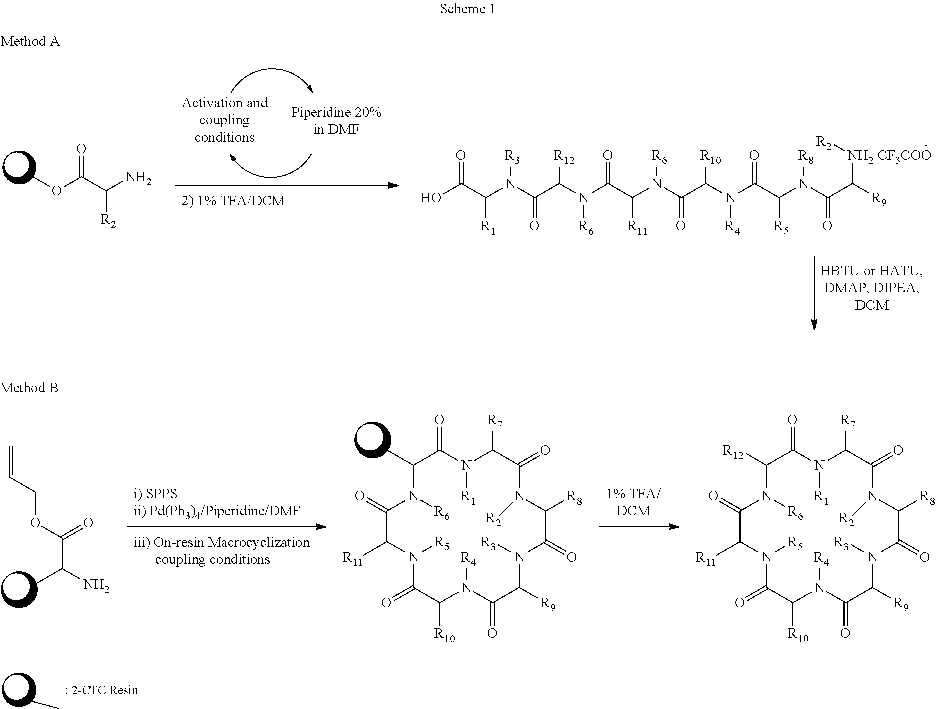

In a second aspect, the present invention provides a pharmaceutical composition for the treatment of malaria, containing a cyclic hexapeptide of formula (I), or any salt, solvate, prodrug, stereoisomer, tautomer thereof, and one or more pharmaceutical acceptable carriers, diluents or excipients. In a preferred embodiment, said pharmaceutical composition contains the cyclic hexapeptide with the general formula (I),

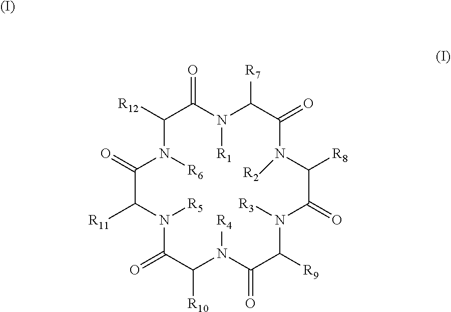

or any salt, solvate, prodrug, stereoisomer, tautomer thereof, wherein
R₁, R₂, R₃, R₄, R₅ and R₆ are independently selected from methyl (—CH₃) and hydrogen (—H)
R₇ is hydrogen (—H), CH₃ or isopropyl.

R₈ and R₁₀ are independently selected from CH₂SC(C₆H₅)₃, CH₂SCH₃, CH₂SH, CH₂S—SR₁₃ (R₁₃═CH₃ or cysteine derived), represented by the formulas:

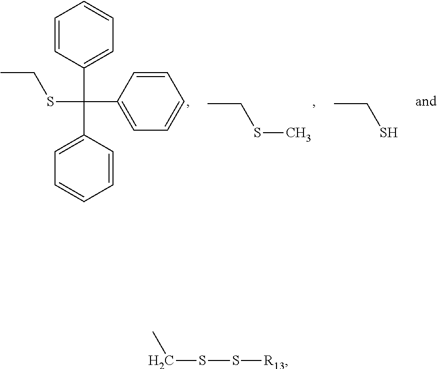

R₉ and R₁₁ are independently selected from CH(CH₃)CH₂CH₃, CH₂C₆H₅, CH₃, CH₂CH₂SCH₃ and H, represented by the formulas:

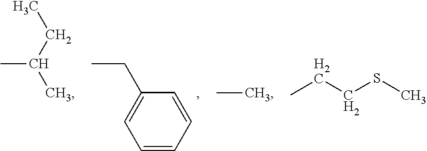

and H′ respectively
R₁₂ is selected from CH₂OH, CH(CH₃)OH, CH₂OC(CH₃)₃, (CH₂)₂COOH, CH(CH₃)OC(CH₃)₃, (CH₂)₂COOR₁₄ represented by the formulas:

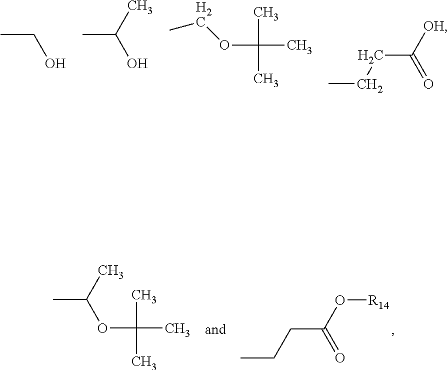

respectively
wherein R₁₄ is an alkyl group (example: CH₃, —CH₂CH₃, n-butyl, C(CH₃)₃, n-propyl, CH(CH₃)₂, etc.).

In one embodiment, the present invention provides cyclic hexapeptides according to the formula (I), wherein at least one of R₁, R₂, R₃, R₄, R₅ or R₆ is a methyl group (—CH₃).

Exemplary cyclic hexapeptide compounds which shown acceptable anti-malarial activity are the following:

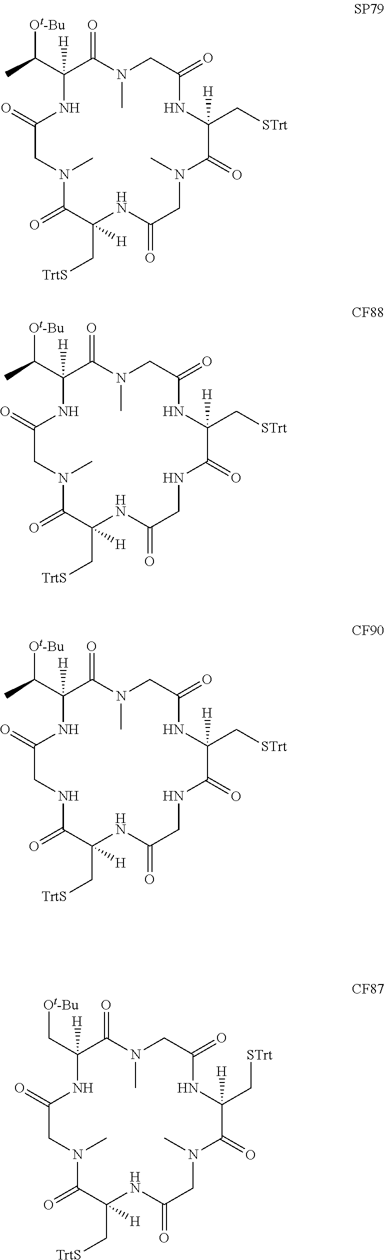

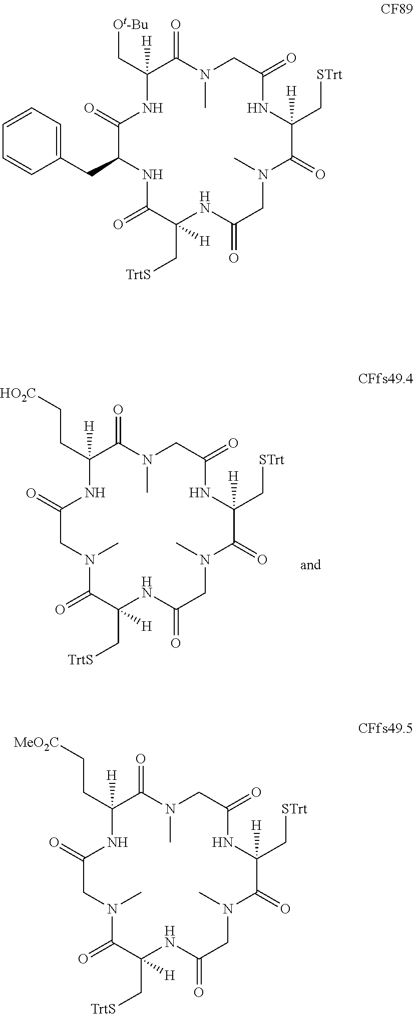

or any salt, solvate, prodrug, stereoisomer, tautomer thereof.

The pharmaceutical acceptable salts of the inventive compounds are typically formed by reacting a compound of a cyclic hexapeptide according to the general formula (I) with an equimolar or excess amount of base. The reactants are generally combined in a mutual solvent such as diethylether, tetrahydrofuran, methanol, ethanol, isopropanol, benzene, and the like for acid addition salts, or water, an alcohol or a chlorinated solvent such as methylene chloride for base

addition salts. It should be recognized that the particular counterion forming a part of any salt of this invention is not of a critical nature, as long as the salt as a whole is pharmacologically acceptable and as long as the counterion does not contribute to undersired qualities of the salt as a whole.

In a further preferred embodiment, the present invention provides cyclic peptides according to the formula (I), which is CF88 represented by the formula:

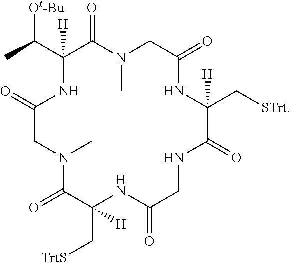

In a further preferred embodiment, the present invention provides cyclic peptides according to the formula (I), which is CFfs49.4 represented by the formula:

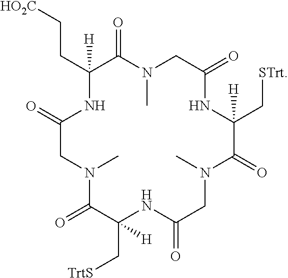

The present pharmaceutical compositions are prepared by well-known procedures in the technical field, using known and readily available ingredients.

In a third aspect, the present invention provides a method for the treatment of malaria, comprising administering to a patient an effective amount of a pharmaceutical composition containing a cyclic hexapeptide according to the general formula (I) or any salt, solvate, prodrug, stereoisomer, tautomer thereof. The composition comprises from 0.1% to 99.9% by weight of said active ingredient.

In a preferred embodiment, said pharmaceutical composition administered to a patient contains the cyclic hexapeptide with the general formula (I), which is selected from the following compounds:

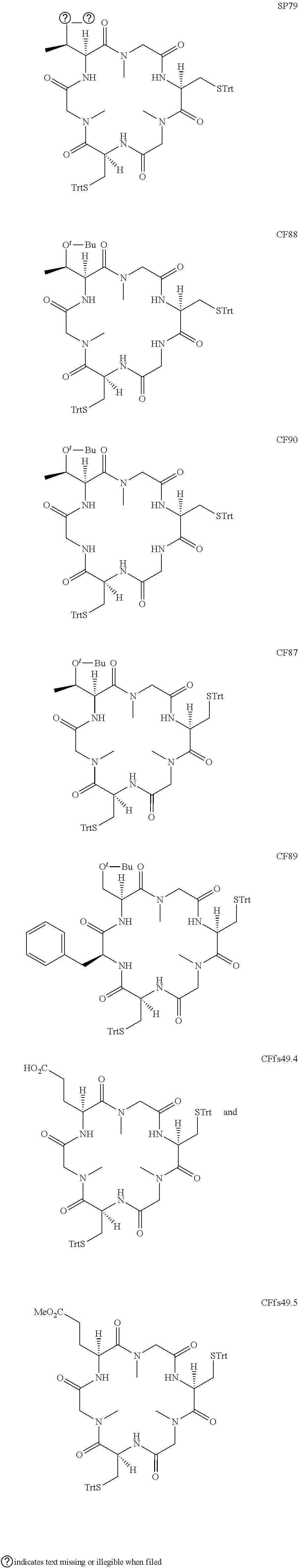

or any salt, solvate, prodrug, stereoisomer, tautomer thereof.

In a preferred embodiment, the pharmaceutical composition administered to a patient for the treatment of malaria contains the compound CF88, represented by the formula:
[symbol] ferred embodiment, the pharmaceutical composition administered [symbol] itment of malaria contains the compound CFfs49.4, represented by

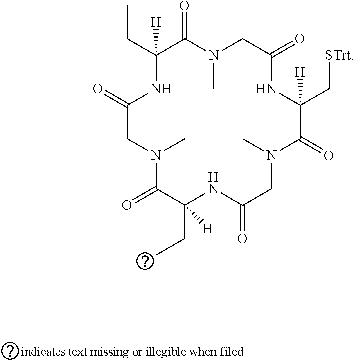

The dose of the pharmaceutical composition containing a cyclic hexapeptide according to the general formula (I) or any salt, solvate, prodrug, stereoisomer, tautomer thereof will vary depending on factors as the nature and severity of the infection, the age and general health of the patient and the tolerance of the patient to the anti-malarial agent. The particular dose regimen likewise may vary according to such factors and thus the compounds may be given in a single daily dose or in multiple doses during the day. The regime may last from about 2-3 days to about several weeks or longer. Without limiting the scope of the invention, the pharmaceutical composition can be administered to the patient preferably for at least 3 days.

The pharmaceutical composition previously described, could be administered through an intramuscular injection, subcutaneously, intravenously, intradermal, intraperitoneal, or may be administered through nasal or oral routes. The preferred route of administration of the pharmaceutical composition of the present invention is orally, for which the active compound is filled into capsules or tablets with suitable excipients, or may be formulated into a flavored liquid suspension, solution or emulsion.

The following examples are intended to illustrate the invention and its preferred embodiments, but they should not be considered under any circumstances to restrict the scope of the invention, which is determined by the content of the claims attached hereto.

### EXAMPLES

### Example 1. Synthesis of the Cyclic Hexapeptide Compounds

The synthesis of the cyclic hexapeptide compounds were performed as disclosed by Fagundez and co-workers (Fagundez C, Sellanes D, Serra G. Synthesis of Cyclic Peptides as Potential Anti-Malarials. ACS Comb Sci, 2018). Briefly, the SPPS general procedure for elongation of the peptide chain was done in a plastic syringe equipped with Teflon filters. The 2-CTC resin (100-300 mesh, 1.20 mmol/g) was swelled in CH₂Cl₂ (3×s). A solution of the first protected amino acid (Fmoc-AA-OH 2.0 equiv) in CH₂Cl₂ and DIPEA (3.0 equiv) was gently shaken for 10 min. Then, an extra 7.0 equiv of DIPEA was added, and shaking was continued for 45 min. MeOH (0.8 mL/g of resin) was then added in order to cap unreacted functional groups on the resin; the mixture was then shaken for 10 min. The resin was filtered and then washed with CH₂Cl₂ (×3), DMF (×3), and CH₂Cl₂ (×3). The N-terminus was deprotected using 20% piperidine in DMF (2×5 min and 1×10 min). The resin was then washed with DMF (×3), CH₂Cl₂ (×3), DMF (×3). A solution of Fmoc-AA-OH (3.0 equiv) and DIPEA (6 equiv) in DMF was added to the resin, followed by a solution of HBTU or HCTU (2.9 equiv) in DMF; or, if the amino acid is Fmoc-Cys(Trt)-OH, a solution of Fmoc-AA-OH (3.0 equiv), DIC (2.9 equiv), and CI-HOBt (2.9 equiv) in DMF was added to the resin. The mixture was stirred for 90 min. After the coupling was completed, the resin was washed with DMF (×3) and CH₂Cl₂ (×3), Deprotection and coupling cycles were repeated with the appropriate amino acids to provide the desired compound. The peptide was cleaved from the resin by treatment with 1% TFA in CH₂Cl₂ for 2-3 min at room temperature followed by filtration and collection of the filtrate in MeOH. The treatment was repeated 3 times and then the resin washed with CH₂Cl₂ (×5). Solvents were removed in vacuo to obtain the crude peptide.

The macrocyclization reaction in solution phase was performed in dilute conditions (1-5 mM) using HBTU or HATU (1.5 equiv), DIPEA (3 equiv), and 4-DMAP (catalytic) in dried CH₂Cl₂ at room temperature over 1-3 days. The reaction mixture was washed with HCl 5% and saturated aqueous NaHCO₃, dried over MgSO₄, filtered, and concentrated in vacuo. The crude was purified by flash chromatography to obtain the macrocycle.

The macrocyclization reaction on-resin was performed using DIC (4 equiv), CI-HOBt (4 equiv), and 4-DMAP (catalytic) in dried DMF/CH₂Cl₂ (8:2) at room temperature overnight. After the macrocyclization was completed, the resin was filtered and then washed with DMF (×3) and CH₂Cl₂ (×3). The macrocycle was cleaved from the resin by treatment with 1% TFA in CH₂Cl₂ for 2-3 min at room temperature followed by filtration and collection of the filtrate in MeOH. The treatment was repeated 3 times and then the resin washed with CH₂Cl₂ (×5). Solvents were removed in vacuo to obtain the crude macrocycle.

### Example 2. Spectroscopic Characteristics of the Exemplary Compounds

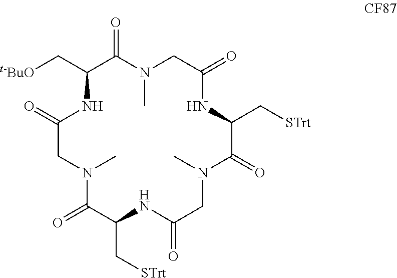

Cycle-NMe-Gly-L-Cys(Trt)-NMe-Gly-L-Ser(^{t}-Bu)-NMe-Gly-L-Cys(Trt) (CF87):

White solid (74%). Rf=0.5 (AcOEt). [α]_{D}²⁵=+74.0 (c 0.99, DCM). ¹H NMR (400 MHz, (CDCl₃) δ (ppm): 1.14 (s, 9H), 2.46-2.60 (m, 1H), 2.64-2.73 (m, 1H), 2.75-3.07 (m, 10H), 3.11-3.29 (m, 4H), 3.33-3.47 (m, 1H), 3.51-3.72 (m, 1H), 4.41-4.60 (m, 3H), 4.65-4.81 (m, 2H), 4.83-4.94 (m, 1H), 7.02-7.59 (m, 33H). ¹³C NMR (100 MHz, (CDCl₃) δ (ppm): 27.3 (3C) 34.2, 37.0, 38.6 (3C), 48.6 (2C), 50.0, 53.4 (2C), 53.9, 63.0, 67.1 (2C), 73.8, 126.9 (6C), 128.0 (12C), 129.7 (12C), 144.4 (6C), 167.4, 167.5, 167.7, 170.5 (2C), 170.8. HRMS m/zcalc. para C₆₀H₆₆N₆O₇S₂ ([M+Na]⁺) 1069.4325, found 1069.4327.

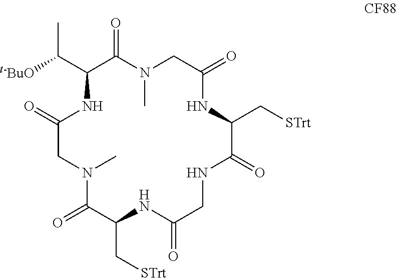

Cycle-NMe-Gly-L-Thr(^{t}-Bu)-NMe-Gly-L-Cys(Trt)-Gly-L-Cys(Trt) (CF88):

White solid (42%). Rf=0.5 (AcOEt). [α]_{D}²⁵=+85.9 (c 1.43, DCM). ¹H NMR (400 MHz, (CD₃)₂CO) δ (ppm): 1.13 (s, 9H), 1.21 (d, J=5.8, Hz, 3H), 2.58-2.70 (m, 1H), 2.65-2.72 (m, 4H), 2.95 (s, 3H), 3.34 (s, 3H), 3.60-3.73 (m, 2H), 3.76-3.85 (m, 1H), 3.97-4.04 (m, 1H), 4.14-4.23 (m, 2H), 4.38-4.48 (m, 2H), 4.54-4.62 (m, 2H), 7.24-7.45 (m, 30H), 7.52-7.58 (m, 1H), 7.63-7.68 (m, 1H), 7.90-7.95 (m, 1H), 7.96-8.02 (m, 1H). ¹³C NMR (100 MHz, (CD₃)₂CO) δ(ppm): 20.3, 27.2 (3C), 31.6, 32.8, 37.9 (2C), 42.4, 51.0, 52.1, 52.3, 53.5, 56.6, 65.9, 66.9, 67.2, 74.2, 127.0 (3C), 127.1 (3C), 128.1 (12C), 129.4 (6C), 129.5 (6C), 144.3 (6C), 165.1, 170.2, 170.5, 171.5, 171.7, 172.0. HRMS m/zcalc. para C₆₀H₆₆N₆O₇S₂ ([M+Na]⁺) 1069.4306, found 1069.4327.

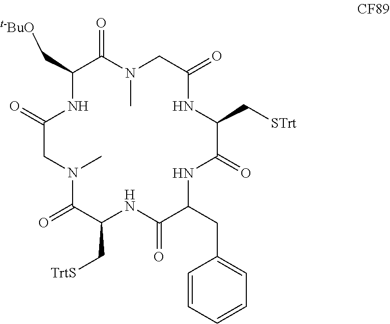

Cycle-NMe-Gly-L-Cys(Trt)-NMe-Gly-L-Cys(Trt)-L-Phe-L-Ser(^{t}-Bu) (CF89):

White solid (57%). Rf=0.4 (AcOEt). [α]_{D}²⁵=−24.3 (c 2.40, DCM). ¹H NMR (400 MHz, (CD₃)₂CO) δ (ppm): 1.06 (s, 9H), 2.47-2.60 (m, 3H), 2.86-3.02 (m, 2H), 2.99 (s, 3H), 3.07-3.23 (m, 2H), 3.18 (s, 3H), 3.39-3.50 (m, 2H), 3.65 (dd, J=5.0, 9.3 Hz, 1H), 4.07-4.15 (m, 1H), 4.16-4.24 (m, 1H), 4.30 (d, J=14.4 Hz, 1H), 4.50 (dd, J=8.1 Hz, 1H), 4.62-4.70 (m, 1H), 4.90 (d, J=16.7 Hz, 1H), 7.11-7.47 (m, 35H), 7.53 (d, J=4.9 Hz, 1H), 7.69 (d, J=9.1 Hz, 1H), 7.76 (d, J=8.2 Hz, 1H), 7.88 (d, J=9.0 Hz, 1H). ¹³C NMR (100 MHz, (CD₃)₂CO) δ(ppm): 26.8 (3C), 32.8, 32.9, 35.6, 36.4, 38.4, 47.44, 51.2, 52.8, 53.3, 53.4, 53.6, 61.8, 66.5, 66.7, 72.7, 126.3, 126.8 (3C), 126.8 (3C), 128.0 (12C), 128.2 (2C), 129.4 (2C), 129.5 (6C), 129.6 (6C), 137.7, 144.6 (3C), 144.9 (3C), 167.3, 168.8, 169.6, 169.9, 171.0, 171.5. HRMS m/zcalc. para C₆₆H₇₀N₆O₇S₂ ([M+Na]⁺) 1146.4640, found 1146.4666.

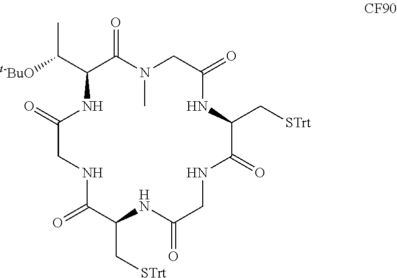

Cycle-L-Thr(^{t}Bu)-NMe-Gly-L-Cys(Trt)-Gly-L-Cys(Trt)-Gly(CF90):

White solid (77%). Rf=0.5 (AcOEt). [α]_{D}²⁵=+20.5 (c 1.9, DCM). ¹H NMR (400 MHz, (CD₃)₂CO) δ (ppm): 0.99 (d, J=6.3, Hz, 3H), 1.23 (s, 9H), 2.47-2.56 (m, 1H), 2.65-2.72 (m, 1H), 2.76-2.84 (m, 1H), 3.07-3.15 (m, 1H), 3.19-3.26 (m, 1H), 3.28 (s, 3H), 3.46-3.55 (m, 1H), 3.57-3.66 (m, 1H), 3.71-3.74 (m, 1H), 3.91-4.03 (m, 2H), 4.04-4.17 (m, 2H), 4.35-4.43 (m, 1H), 4.67 (t, J=5.4 Hz, 1H), 6.09-6.17 (m, 1H), 6.18-6.26 (m, 1H), 6.67-6.76 (m, 1H), 7.08-7.16 (m, 1H), 7.19-7.37 (m, 21H), 7.38-7.50 (m, 12H). ¹³C NMR (100 MHz, (CD₃)₂CO) δ(ppm): 18.5, 28.2 (3C), 31.8, 32.8, 39.2, 43.0, 43.1, 52.2, 53.3, 54.1, 54.7, 67.4 (2C), 67.7, 75.1, 127.0 (6C), 128.1 (12C), 129.5 (12C), 144.3 (3C), 144.4 (3C), 168.7, 168.9, 169.4, 170.4, 170.5, 170.8. HRMS m/zcalc. para C₅₉H₆₄N₆O₇S₂ ([M+Na]⁺) 1055.4170, found 1055.4148.

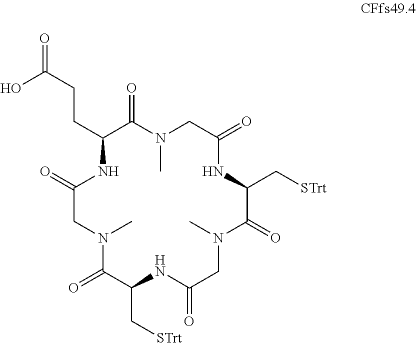

Cycle-NMe-Gly-L-Cys(Trt)-NMe-Gly-L-Cys(Trt)-NMe-Gly-L-Glu (CFfs49.4):

Yellow solid (84%). t_{R}=9.40 min (linear gradient: 50 a 100% acetonitrile: H₂O/0.003M de TFA over 10 min; flujo=1.5 ml/min). ¹H NMR (400 MHz, DMSO) δ (ppm): 2.31-2.39 (m, 2H), 2.39-2.41 (m, 2H), 2.54-2.74 (m, 4H), 2.82-2.91 (m, 9H), 3.29-3.54 (m, 2H), 3.75-3.88 (m, 1H), 4.17-4.32 (m, 1H), 4.36-4.52 (m, 2H), 4.64-4.86 (m, 3H), 4.86-5.06 (m, 1H), 7.18-7.47 (m, 33H). ¹³C NMR (100 MHz, DMSO) δ(ppm): 22.7, 29.5, 35.0 (2C), 38.1, 47.4, 48.4 48.5, 51.3, 51.7 (2C), 66.2, 66.5, 127.1, 128.0, 128.2, 129.6, 144.7, 145.0, 169.9, 170.0 (2C), 170.6, 171.0, 172.0, 174.4. HRMS m/zcalc. for C₅₈H₆₀N₆O₈S₂ ([M+Na]⁺) 1055.38. found 1055.26; ([M+H]⁺) 1033.27, found 1033.28.

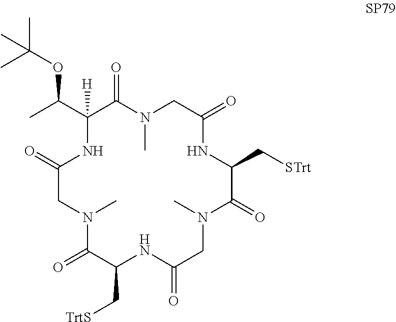

Cycle-NMe-Gly-L-Thr(^{t}-Bu)-NMe-Gly-L-Cys(Trt)-NMe-Gly-L-Cys(Trt) (SP79):

White solid (35%). Rf=0.55 (AcOEt:EP, 4:1). ¹H NMR (400 MHz, CDCl₃) δ ppm 1.02 (d, J=6.4 Hz, 3H), 1.24 (s, 9H), 2.42-2.53 (m, 2H), 2.60-2.71 (m, 2H), 2.80 (s, 3H), 2.82 (s, 3H), 2.95-3.16 (m, 3H), 3.27 (s, 3H), 3.93 (dd, J=6.4 Hz, J=3.8 Hz, 1H), 4.54 (d, J 450=13.6 Hz, 1H), 4.63 (d, J=13.2 Hz, 1H), 4.64 (d, J=13.4 Hz, 1H), 4.72-4.80 (m, 3H), 7.14-7.63 (m, 30H), 7.54-7.63 (m, 7.67, 2H), 7.67 (d, J=6.8 Hz, 1H). ¹³C NMR (100 MHz, CDCl₃) δ ppm 18.0, 28.0, 34.4, 36.6, 36.9, 38.4, 48.5, 53.3, 53.8, 54.0, 54.5, 66.9, 68.2, 75.5, 126.8, 128.0, 129.6, 144.4, 167.1, 167.4, 167.6, 169.8, 170.2, 170.3. HRMS calculated for C₆₁H₆₈N₆NaO₇S₂ ([M+Na]⁺) 1083.4489, found 1083.4447.

### Example 3. Structures of Cyclic Hexapeptide Compounds and Biological Activities In Vitro

For the in vitro study of the antimalarial activity against P. falciparum K1, this strain originating from Thailand that is resistant to chloroquine and pyrimethamine but sensitive to mefloquine was used. The cultures are naturally asynchronous (65-75% ring stage) and are maintained in continuous log phase growth in RPM11640 medium supplemented with 5% washed human A+erythrocytes, 25 mM HEPES, 32 nM NaHCO₃, and AlbuMAXII (lipid-rich bovine serum albumin) (GIBCO, Grand Island, N.Y.) (CM). All cultures and assays are conducted at 37° C. under an atmosphere of 5% CO₂ and 5% 02, with a balance of N₂.

For drug sensitivity assays, tock drug solutions are prepared in 100% DMSO (dimethylsulfoxide) at 20 mM. The compound is further diluted to the appropriate concentration using complete medium RPM11640 supplemented with 15 nM cold hypoxanthine and AlbuMAXII. Assays are performed in sterile 96-well microtitre plates, each plate contains 100 mL of parasite culture (0.5% parasitemia, 2.5% hematocrit). Each drug is tested in triplicate and parasite growth is compared to control and blank (uninfected erythrocytes) wells. After 24 h of incubation at 37° C., 3.7 Bq of [3H]hypoxanthine is added to each well. Cultures are incubated for a further 24 h before they are harvested onto glass-fiber filter mats. The radioactivity is counted using a Wallac Microbeta 1450 scintillation counter. The results are recorded as counts per minute (CPM) per well at each drug concentration, control and blank wells. The percentage inhibition is calculated from comparison of blank and control wells, and EC₅₀ values are calculated using Prism™. Screen. The K1 line is used. The compound is diluted threefold over 12 different concentrations with an appropriate starting concentration. The EC₅₀ is determined by a sigmoidal dose-response analysis using Prism™. For each assay, the EC₅₀ value for the parasite line is determined against the known antimalarials chloroquine and artesunate, plus other standard compounds appropriate for the assay.

For the in vitro study of the antimalarial activity against P. falciparum D7, two stock solutions were prepared: solution A (dilution of powder sample) at 20 mM and solution B at 1 mM (made by dilution of solution A). Next, the compounds were diluted 100-fold (drug solution=10 μM), then, suitable volume of drug solution was transferred in the SYBR GREEN assay plate (final concentration=10-0.15 μM). However, due to the high potency of the samples, the initial concentration was reduced in order to determine the EC₅₀ values. The assay condition included: hematocrit at 2%, initial parasitemia 0.5% and artesunate and pyrimethamine were used as positive control. The fluorescence of each well is measured. The EC₅₀ value of each compound was assessed in at least two independent experiments.

Cyclic hexapeptide compounds represented by the following formulas:

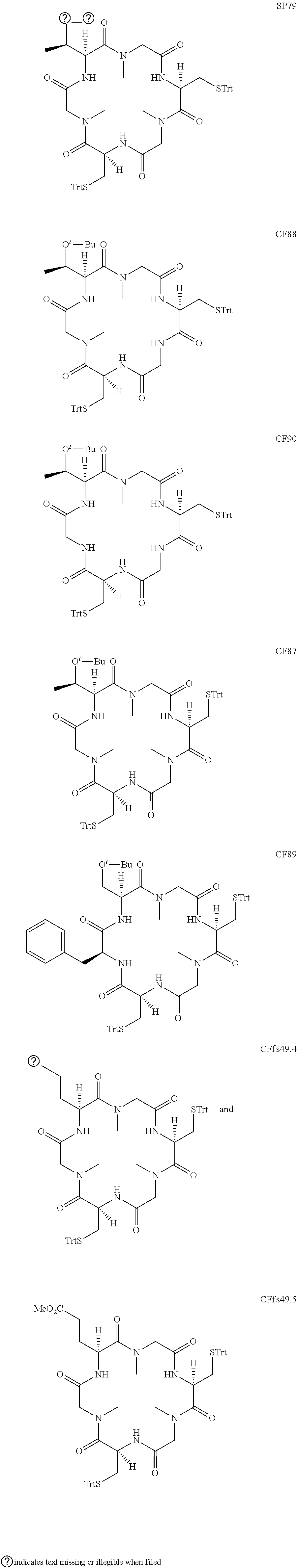

were tested in vitro against the intraerythrocytic form of the parasite Plasmodium falciparum K1 (chloroquine-resistant strain) and Plasmodium falciparum 31D7 (chloroquine-sensitive strain). The selectivity index (SI) was determined as the ration between EC₅₀ against murine macrophages or HepG2 cells and the EC₅₀ against P. falciparum. In the following Table 1 is shown the results of the EC₅₀ obtained for each of the tested compounds against P. falciparum K1 (P. f. K1) and P. falciparum 3D7 (P. f. 3D7) and the SI calculated. Also, Table 1 shows the results of the biological activity in vitro of two compounds (SP79 and CF89) against the hepatic form of the parasite.

TABLE 1
Biological activities in vitro of cyclic hexapeptides against
P. falciparum

| | EC₅₀P. f. | EC₅₀P. f. | | EC₅₀P. b. |
|---|---|---|---|---|
| | K1^{a} | 3D7^{b} | | (liver st) |
| Compound | (nM) | (nM) | SI | (μM) |
| SP79 | 0.7 | 1.0 | >140000 | 0.0186 |
| 3.8 > 12500- | 0.008 | 0.25 | >1 × 10⁶ | — |
| CF88 | | | | |
| CF87nt | 0.13 | 1.4 | >1 × 10⁵ | — |
| CF90 | | | | |
| CF89 | 9.0 | 1.8 | >1 × 10⁵ | 0.335 |
| CFfs49.4 | 0.2 | 12 | >20000 | — |
| CFfs49.5 | nt | 5.2 | >40000 | — |

^{a}Control: Chloroquine, EC₅₀ = 470 nM, Artemisinine: EC₅₀ = 20 nM, Artesunate: EC₅₀ = 3 nM;
^{b}Control: Pyrimethamine EC₅₀ = 31 nM, Artesunate: EC₅₀ = 5 nM nt: not tested

Additionally, killing rates assays were performed, which contribute to elucidate the mode-of-action of the cyclic hexapeptide compounds against the parasites (Sanz L M, Crespo B, De-Cózar C, Ding X C, Llergo J L, Burrows J N, Garcia-Bustos J F, Gamo F J. P. falciparum in vitro killing rates allow discriminating between different antimalarial mode-of-action. PloS One, 2012, 7(2):e30949), and the data obtained were also useful to determine the appropriate administration route of the pharmaceutical composition that contains a cyclic hexapeptide compound (time intervals and doses). The assay was performed in P. falciparum 3D7 strain in its intraerythrocytic form. The parasite survival percentage was determined at 24 and 48 hrs. Each compound was renewed (washing the parasites and adding culture media and the compound again) after the first 24 hours of treatment. The compound concentration was calculated as 10×EC₅₀ executed by the inventors: for compound CF89 500 nM and for SP79 30 nM. The culture media used was RPMI 1640 25 nM HEPES and NaHCO₃ supplemented with 2% D-sucrose, 0.3% L-glutamine, 0.150 hypoxanthine and AlbuMAX II solution 5 g/L. Table 2 shows the results of the killing rates assays for compounds CF89 and CFfs49.4.

TABLE 2
Killing rates for P. f. 3D7 and survival percentage according to the
times of treatment

| | Time of Treatment | | | | |
|---|---|---|---|---|---|
| | 0 hrs | 24 hrs | | 48 hrs | |
| | Survival | Survival | Standard | Survival | Standard |
| Compound | % | % | Deviation | % | Deviation |
| CF89 | 100 | 47.83 | 5.88 | 26.01 | 6.02 |
| SP79 | 100 | 83.74 | 7.58 | 57.48 | 0.15 |
| Artesunate | 100 | 10.01 | 2.09 | 8.59 | 1.23 |
| Chloroquine | 100 | 14.13 | 4.84 | 7.04 | 3.07 |
| Pyrimethamine | 100 | 53.52 | 1.36 | 9.59 | 2.62 |
| Atovaquone | 100 | 56.55 | 4.36 | 32.02 | 6.40 |

### Example 4. In Vivo Assays for Cyclic Hexapeptide Compounds

The most promising cyclic hexapeptide compounds (CF88 and CFfs49.4) were tested in vivo in mice inoculated with P. berghei. Each mouse was treated with 50 mg/kg through oral route of administration for 3 days (24 hrs, 48 hrs and 72 hrs after infection). Table 3 shows the results of these assays.

TABLE 3
Anti-malarial activity in vivo in mice inoculated with P. berghei,
treated 3 consecutive days

| Compound | Parasitemia (reduction %) | | | Days of |
|---|---|---|---|---|
| 50 mg/Kg | Day 5 | Day 7 | Day 9 | survival |
| CF88 | 0.9 ± 0.7 | 1.7 ± 0.3 | 6 ± 1 | 25 ± 7 |
| | (70 ± 24%) | (46 ± 10%) | (33 ± 10%) | |
| CFfs49.4 | 1.1 ± 0.2 | 1.6 ± 0.2 | 4.8 ± 0.5 | 23 ± 4 |
| | (66 ± 5%) | (46 ± 6%) | (50 ± 4%) | |
| Chloroquine | 0 | 0 | 0 | >30 |
| | (100%) | (100%) | (100%) | |
| No Treatment | 3.2 ± 0.5 | 3.1 ± 0.4 | 9 ± 2 | 23 ± 6 |

As shown in Table 3, the cyclic hexapeptides CF88 and CFfs49.4 were active in vivo, reducing the parasitemia measured on day 5 (96 hrs after infection) at 70% and 66%, respectively. Further studies will be carried out to improve these results to achieve parasitemia with the compounds at the desired levels.

### Example 5. Pharmacokinetics Properties of Cyclic Hexapeptide Compounds

The objective of this study was to investigate the plasma pharmacokinetics of the compound CF-88 in male Swiss Albino mice following a single oral administration. Nine animals were used in this study as Group 1 (PO: 50 mg/kg). Animals were administered with solution formulation of CF-88 in 3% DMSO and RPMI medium via oral route at 50 mg/kg. The blood samples were collected from set of three mice at each time point in labeled micro centrifuge tube containing K2EDTA solution as anticoagulant at 0.25, 0.5, 1, 2, 4, 6, 8, 12 and 24 hr. Plasma samples were separated by centrifugation of whole blood and stored below −70±10° C. until bioanalysis. All samples were processed for analysis by protein precipitation using acetonitrile and analyzed with fit-for-purpose LC-MS/MS method (LLOQ=5.16 ng/mL). Pharmacokinetic parameters were calculated using the non-compartmental analysis tool of Phoenix WinNonlin® (Version 7.0).

For the preparation of the oral formulation accurately weighed 19.93 mg of compound for PO dosing was added in a labeled bottle. The volume 0.112 mL of DMSO was added and vortexed. The volume 3.617 mL of RPMI medium was added and vortexed. The final formulation (5 mg/mL) was vortexed for 2 minutes to get finely suspended formulation.

After preparation of formulations, a volume of 200 μL was aliquoted for analysis. The formulations were found to be within the acceptance criteria (in-house acceptance criteria is ±20% from the nominal value). Formulations were prepared freshly prior to dosing. Following oral administration of CF-88 at 50 mg/kg dose, all animals were normal without any clinical signs.

Blood samples (approximately 60 μL) were collected from retro-orbital plexus of three mice at 0.25, 0.5, 1, 2, 4, 6, 8, 12 and 24 hr. Samples were collected into labeled micro-tubes, containing K2EDTA solution (20% K2EDTA solution) as an anticoagulant. Plasma was immediately harvested from the blood by centrifugation at 4000 rpm for 10 min at 4±2° C. and stored below −70° C. until bioanalysis.

The results of these assays are shown in the following Table 4 and the sole FIGURE. This assayed compound had a maximum concentration in plasma at 2 hrs and it was detected up to 12 hrs after the administration, with no detection in plasma at 24 hrs. The concentration at 12 hrs was 10.78 ng/mL or 10 nM, above the EC₉₀ for CF88 (1 nM against P. falciparum K1). In consequence, there was enough concentration of CF88 compound in plasma up to 12 hrs from the administration (minimum observed concentration) to eliminate at least the 90% of parasites. Mean pharmacokinetic parameters are summarized below:

TABLE 4
Pharmacokinetic properties in plasma of CF88

| | | | | | AUCₗₐₛₜ | AUC_{inf} | |
|---|---|---|---|---|---|---|---|
| | | Dose | tₘₐₓ | Cₘₐₓ | (hr*ng/ | (hr*ng/ | t_{1/2} |
| Compound | Route | (mg/kg) | (hr) | (ng/mL) | mL) | mL) | (hr) |
| CF-88 | PO | 50 | 2.00 | 61.13 | 314.79 | 390.71 | 4.93 |

Note:
For calculation of T_{1/2}, 4, 6, 8 and 12 hr time points were considered (r² = 0.7325)

Cyclic peptides of the present invention possess desirable properties that make them promising candidates for the development of novel drug molecules. In general, they present structural features to favor bioactive conformations, selectivity to the receptors and metabolic stability. In addition, cell permeability and oral bioavailability could be enhanced by controlling hydrophobicity and the number of hydrogen-bond by N-methylation. Based in these concepts, N-methyl amino acids (NMe-AA) were used to prepare cyclopeptides. The compounds have activity against asexual liver and blood stages. This novel chemical series showed EC₅₀ low or sub-nanomolar. In addition, the compounds have the following key features: half-life in rodents=4.93 h and confirmed in vivo efficacy.

In conclusion, the compounds are very promising as new drugs for malaria treatment. It should be understood that after reading its teachings, those skilled in the art could make changes or modifications to the present invention, however, these different forms would be considered within the scope of the invention, which is defined by the attached set of claims.

1. A cyclic hexapeptide compound of the general formula (I): 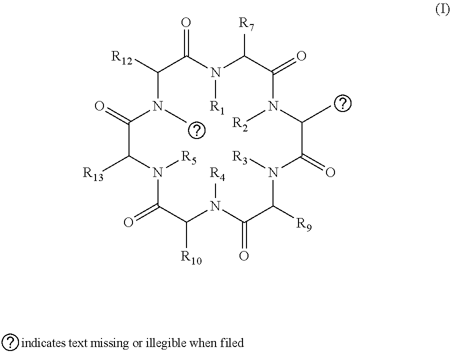
or any salt, solvate, prodrug, stereoisomer, tautomer thereof, wherein
R₁, R₂, R₃, R₄, R₅ and R₆ are independently selected from methyl (—CH₃) and hydrogen (—H);
R₇ is hydrogen (—H), CH₃ or isopropyl.
R₈ and R₁₀ are independently selected from CH₂SC(C₆H₅)₃, CH₂SCH₃, CH₂SH, CH₂S—SR₁₃, wherein R₁₃ is CH₃ or cysteine derived, represented by the formulas: 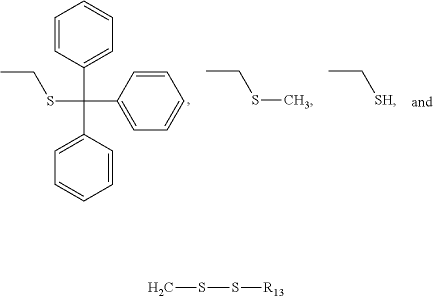
respectively
R₉ and R₁₁ are independently selected from CH(CH₃)CH₂CH₃, CH₂C₆H₅, CH₃, CH₂CH₂SCH₃ and H, represented by the formulas: 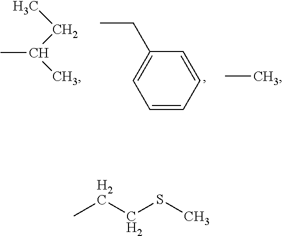
and H, respectively
R₁₂ is selected from CH₂OH, CH(CH₃)OH, CH₂OC(CH₃)₃, (CH₂)₂COOH, CH(CH₃)OC(CH₃)₃, (CH₂)₂COOR₁₄ represented by the formulas: 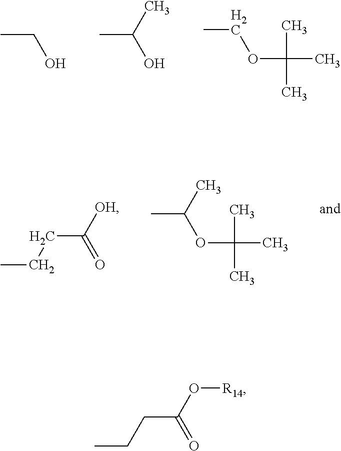
respectively
wherein R₁₄ is an alkyl group selected from CH₃, —CH₂CH₃, n-butyl, C(CH₃)₃, n-propyl, CH(CH₃)₂.

2. The cyclic hexapeptide compound of claim 1, wherein at least one of R₁, R₂, R₃, R₄, R₅ or R₆ is a methyl group (—CH₃).

3. The cyclic hexapeptide compound of claim 2, which is selected from the following compounds: 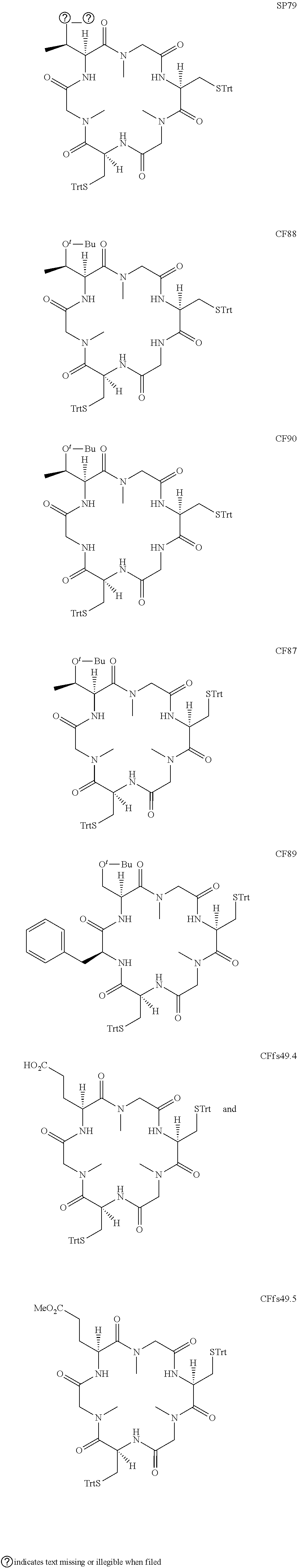
or any salt, solvate, prodrug, stereoisomer, tautomer thereof.

4. The cyclic hexapeptide compound of claim 3, which is the compound CF88 represented by the formula:

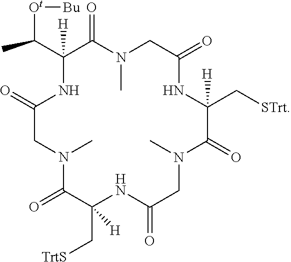

5. The cyclic hexapeptide compound of claim 3, which is the compound CFfs49.4 represented by the formula:

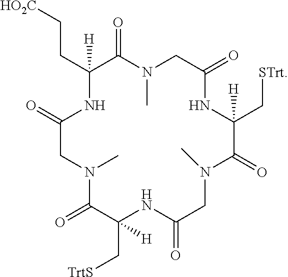

6. A pharmaceutical composition for the treatment of malaria, containing a cyclic hexapeptide compound of general formula (I): 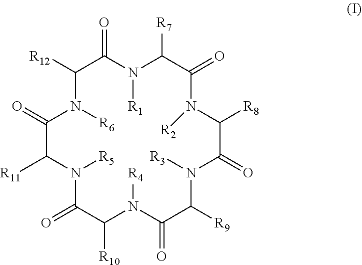
or any salt, solvate, prodrug, stereoisomer, tautomer thereof, wherein
R₁, R₂, R₃, R₄, R₅ and R₆ are independently selected from methyl (—CH₃) and hydrogen (—H);
R₇ is hydrogen (—H), CH₃ or isopropyl.
R₈ and R₁₀ are independently selected from CH₂SC(C₆H₅)₃, CH₂SCH₃, CH₂SH, CH₂S—SR₁₃, where R₁₃ is CH₃ or cysteine derived, represented by the formulas: 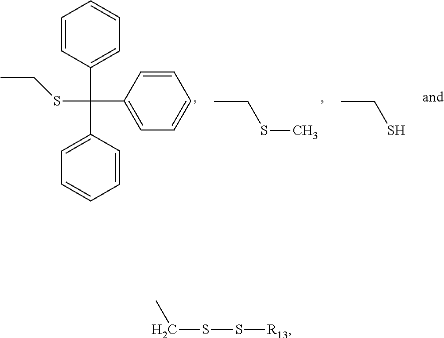
respectively
R₉ and R₁₁ are independently selected from CH(CH₃)CH₂CH₃, CH₂C₆H₅, CH₃, CH₂CH₂SCH₃ and H, represented by the formulas: 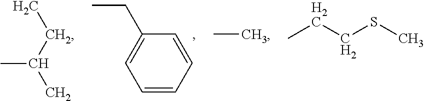
and H, respectively
R₁₂ is selected from CH₂OH, CH(CH₃)OH, CH₂OC(CH₃)₃, (CH₂)₂COOH, CH(CH₃)OC(CH₃)₃, (CH₂)₂COOR₁₄ represented by the formulas: 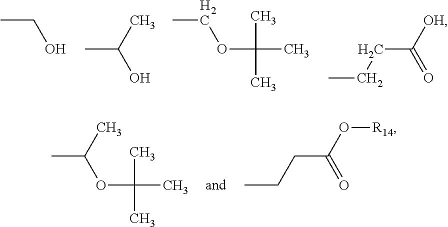
respectively
wherein R₁₄ is an alkyl group selected from CH₃, —CH₂CH₃, n-butyl, C(CH₃)₃, n-propyl, CH(CH₃)₂; and a pharmaceutically acceptable excipient.

7. The pharmaceutical composition for the treatment of malaria of claim 6, wherein at least one of R₁, R₂, R₃, R₄, R₅ or R₆ is a methyl group (—CH₃).

8. The pharmaceutical composition for the treatment of malaria of claim 7, wherein the cyclic hexapeptide compound is selected from the following compounds: 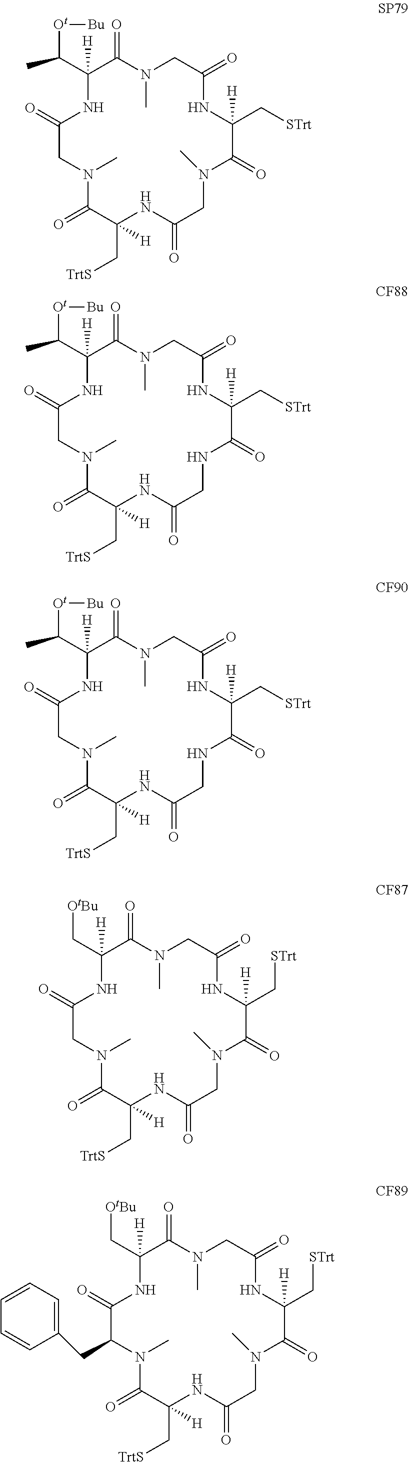 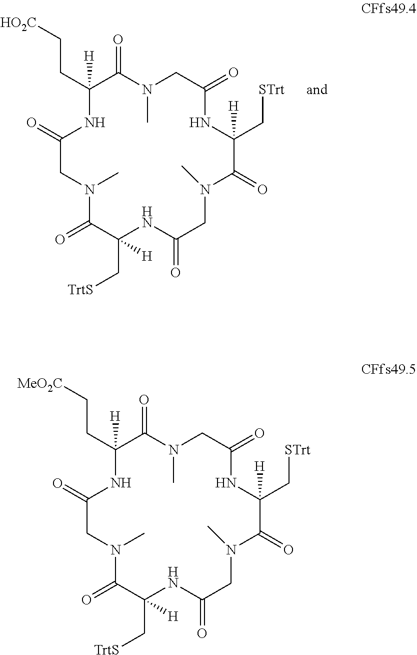
or any salt, solvate, prodrug, stereoisomer or tautomer thereof.

9. The pharmaceutical composition for the treatment of malaria of claim 8, wherein the cyclic hexapeptide compound is the compound CF88 represented by the formula:

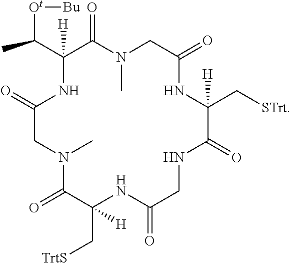

10. The pharmaceutical composition for the treatment of malaria of claim 8, wherein the cyclic hexapeptide compound is the compound CFfs49.4 represented by the formula:

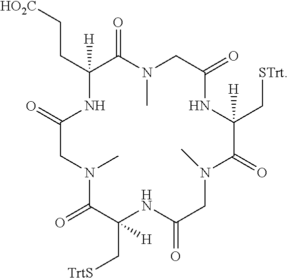

11. A method for the treatment of malaria, comprising the administration to a patient of a pharmaceutical composition containing a cyclic hexapeptide compound of general formula: 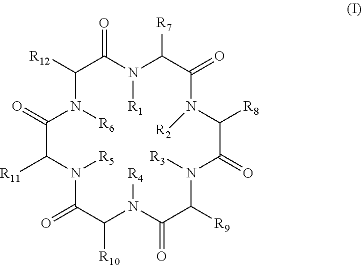
or any salt, solvate, prodrug, stereoisomer, tautomer thereof, wherein
R₁, R₂, R₃, R₄, R₅ and R₆ are independently selected from methyl (—CH₃) and hydrogen (—H);
R₇ is hydrogen (—H), CH₃ or isopropyl.
R₈ and R₁₀ are independently selected from CH₂SC(C₆H₅)₃, CH₂SCH₃, CH₂SH, CH₂S—SR₁₃ wherein R₁₃ is CH₃ or cysteine derived, represented by the formulas: 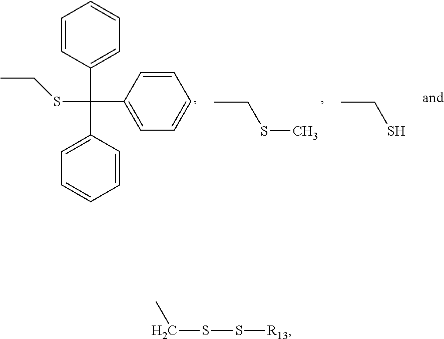
respectively
R₉ and R₁₁ are independently selected from CH(CH₃)CH₂CH₃, CH₂C₆H₅, CH₃, CH₂CH₂SCH₃ and H, represented by the formulas: 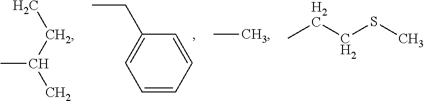
and H′ respectively
R₁₂ is selected from CH₂OH, CH(CH₃)OH, CH₂OC(CH₃)₃, (CH₂)₂COOH, CH(CH₃)OC(CH₃)₃, (CH₂)₂COOR₁₄ represented by the formulas: 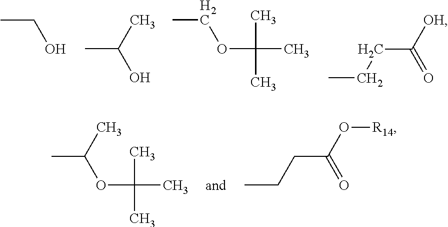
respectively
wherein R₁₄ is an alkyl group selected from CH₃, —CH₂CH₃, n-butyl, C(CH₃)₃, n-propyl, CH(CH₃)₂.

12. The method of claim 11, wherein at least one R₁, R₂, R₃, R₄, R₅ or R₆ is a methyl group (—CH₃).

13. The method of claim 12, wherein the cyclic hexapeptide compound is selected from the following compounds: 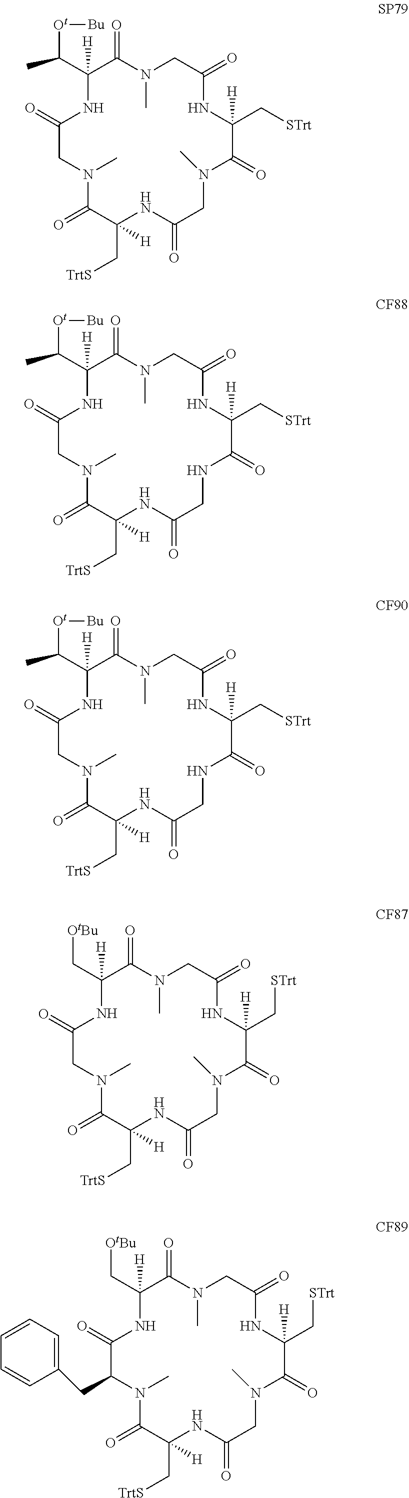 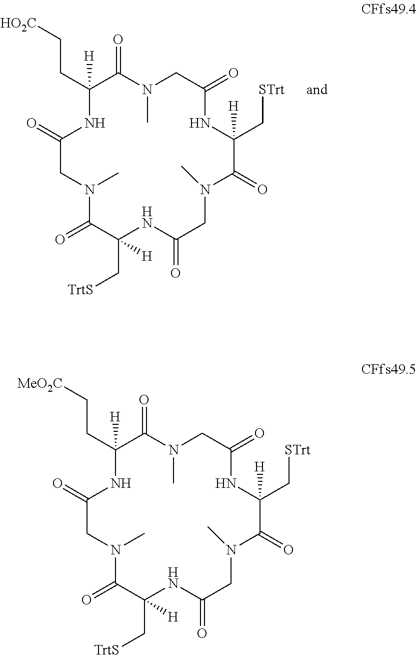
or any salt, solvate, prodrug, stereoisomer or tautomer thereof.

14. The method of claim 13, wherein the cyclic hexapeptide compound is the compound CF88 which is represented by the formula:

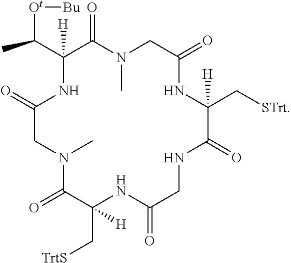

15. The method of claim 13, the cyclic hexapeptide compound is the compound CFfs49.4 which is represented by the formula:

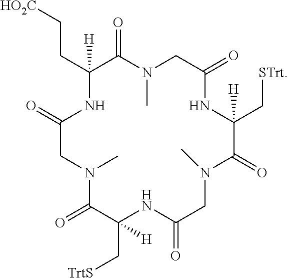

16. The method of the claim 11, wherein said pharmaceutical composition is administered to the patient for at least 3 days.

17. The method of the claim 11, wherein said pharmaceutical composition is administered orally.
